(19) United States
(12) Patent Application Publication
(10) Pub. No.: US 2021/0354135 A1
(43) Pub. Date: Nov. 18, 2021

(54) SYSTEMS AND METHODS FOR EVALUATING AN ABILITY OF A BLOOD SAMPLE TO COAGULATE, CLOT, AND/OR OCCLUDE

(71) Applicant: The Texas A&M University System, College Station, TX (US)

(72) Inventors: Abhishek Jain, Cypress, TX (US); David Jordan Luna, College Station, TX (US)

(73) Assignee: The Texas A&M University System, College Station, TX (US)

(21) Appl. No.: 17/319,918

(22) Filed: May 13, 2021

Related U.S. Application Data
(60) Provisional application No. 63/023,893, filed on May 13, 2020.

(51) Int. Cl. B01L 3/00; G01N 33/49; G01N 33/86; C12Q 1/56

(52) U.S. Cl. CPC B01L 3/502715; B01L 3/50273; G01N 33/4905; G01N 33/86; B01L 2300/0861; B01L 2300/0883; B01L 2400/0475; B01L 2300/0627; C12Q 1/56

(57) ABSTRACT

A system for evaluating an ability of a blood sample to coagulate, clot, and/or occlude includes a microfluidic device including a body and a tortuous microchannel formed in the body, wherein the tortuous microchannel includes a plurality of alternating curves and has a tortuosity index (TI) of at least 2.0, and a pump fluidically configured to fluidically connect to the tortuous microchannel of the microfluidic device and to induce a blood flow through the tortuous microchannel at at least one of a constant pressure and a constant flowrate.

### CROSS-REFERENCE TO RELATED APPLICATIONS

This application claims benefit of U.S. provisional patent application Ser. No. 63/023,893 filed May 13, 2020, and entitled “Microengineered Medical Device Mimicking Tortuous Blood Vessels for Measuring Thrombus, Coagulation, and Platelet Function,” which is hereby incorporated herein by reference in its entirety for all purposes.

### STATEMENT REGARDING FEDERALLY SPONSORED RESEARCH OR DEVELOPMENT

Not applicable.

### BACKGROUND

Prevention of thrombosis with anticoagulation and other forms of blood clot preventing drugs is critical for treatment of many diseases and conditions (e.g., atrial fibrillation, sepsis, trauma, prosthetic heart valves, various coagulopathies or other bleeding disorders) as well as for many life-saving procedures, including dialysis, hemofiltration, extracorporeal oxygenation (ECMO), treatment using a ventricular assist device (VAD), angioplasty, intravenous fluid delivery, apheresis and collection of blood samples for analysis or culture. Additionally, hemostasis monitoring is critically important in pediatric patients on extracorporeal life support systems (ECLS) and receiving anticoagulant and anti-platelet therapies to anticipate, avoid and direct the management of bleeding and thrombotic risk. For example, bleeding and thrombosis episodes in children are often encountered during ECMO and/or VAD. Patients who have bleeding or clotting problems and/or otherwise undergoing a procedure may be monitored using Prothrombin Time (PT) and Activated Clotting Time (ACT)/Activated Partial Clotting Time (APTT) tests, which provide semi-quantitative measures of the extrinsic or intrinsic coagulation pathways respectively.

### BRIEF SUMMARY OF THE DISCLOSURE

An embodiment of a system for evaluating an ability of a blood sample to coagulate, clot, and/or occlude comprises a microfluidic device comprising a body and a tortuous microchannel formed in the body, wherein the tortuous microchannel comprises a plurality of alternating curves and has a tortuosity index (TI) of at least 2.0, and a pump fluidically configured to fluidically connect to the tortuous microchannel of the microfluidic device and to induce a blood flow through the tortuous microchannel at at least one of a constant pressure and a constant flowrate. In some embodiments, the system further comprises a sensor configured to fluidically connect to the tortuous microchannel of the microfluidic device and configured to determine at least one of the pressure and the flowrate induced by the pump, and a controller configured to determine a clotting time of the blood flow based on at least one of the pressure and the flowrate of the blood flow determined by the sensor. In some embodiments, the clotting time determined by the controller corresponds to a time period at which the pressure of the blood flow is between 2.3 times and 3.0 times greater than an initial pressure of the blood flow at an initiation of the blood flow induced by the pump. In certain embodiments, the tortuous microchannel of the microfluidic device is sinusoidal and has a frequency between 2.0 and 2.5 cycles per millimeter (mm). In certain embodiments, the tortuous microchannel of the microfluidic device has an amplitude of between 2 mm and 5 mm. In some embodiments, the tortuous microchannel of the microfluidic device has an arc angle between the alternating curves thereof between 30° and 40°. In some embodiments, the tortuous microchannel has a cross-sectional flow area between 12,000 square micrometers (μm²) and 20,000 μm². In certain embodiments, the microfluidic device further comprises an inlet reservoir and an inlet microchannel extending from the inlet reservoir each formed in the body, and an outlet microchannel extending to an outlet reservoir each formed in the body thereof, wherein the tortuous microchannel is positioned between the inlet microchannel and the outlet microchannel. In certain embodiments, the microfluidic device comprises a plurality of inlet microchannels, tortuous microchannels, and outlet microchannels extending in parallel between the inlet reservoir and the outlet reservoir. In some embodiments, the tortuous microchannel is coated with at least one of a thrombus forming agent, a reagent, collagen, and a thrombus inhibiting agent, a platelet activating material, a platelet inhibiting material, a fibrin network forming material, a fibrin network disrupting material, and/or cells. In some embodiments, the pump is configured to induce the blood flow at a flowrate of between 50 microliters per minute (μl/min) and 100 μl/min.

An embodiment of a system for evaluating an ability of a blood sample to coagulate, clot, and/or occlude comprises a microfluidic device comprising a body and a tortuous microchannel formed in the body, wherein the tortuous microchannel comprises a plurality of alternating curves having an arc angle between the alternating curves between 30° and 40°, and a pump fluidically configured to fluidically connect to the tortuous microchannel of the microfluidic device and to induce a blood flow through the tortuous microchannel at at least one of a constant pressure and a constant flowrate. In some embodiments, the system further comprises a sensor configured to fluidically connect to the tortuous microchannel of the microfluidic device and configured to determine at least one of the pressure and the flowrate induced by the pump, a controller configured to determine a clotting time of the blood flow based on at least one of the pressure and the flowrate of the blood flow determined by the sensor. In some embodiments, the clotting time determined by the controller corresponds to a time period at which the pressure of the blood flow is between 2.3 times and 3.0 times greater than an initial pressure of the blood flow at an initiation of the blood flow induced by the pump. In some embodiments, the tortuous microchannel of the microfluidic device is sinusoidal and has a frequency between 2.0 and 2.5 cycles per millimeter (mm). In certain embodiments, the tortuous microchannel of the microfluidic device has a tortuosity index (TI) of at least 2.0.

An embodiment of a method or evaluating an ability of a blood sample to coagulate, clot, and/or occlude comprises (a) inducing a blood flow by a pump through a tortuous microchannel formed in a body of a microfluidic device, wherein the tortuous microchannel comprises a plurality of alternating curves and has a tortuosity index (TI) of at least 2.0, (b) monitoring at least one of a flowrate and a pressure by a sensor fluidically connected to the tortuous microchannel, (c) determining by a controller a clotting time of the blood flow based on a change in the pressure or the flowrate of the blood flow monitored by the sensor. In some embodiments, the clotting time corresponds to a time period at which the pressure of the blood flow is between 2.3 times and 3.0 times greater than an initial pressure of the blood flow at an initiation of the blood flow induced by the pump. In some embodiments, (b) comprises inducing the blood flow by the pump through the tortuous microchannel at a flowrate of between 50 microliters per minute (μl/min) and 100 μl/min such that a shear rate gradient of greater than 5000 per second per millimeter (1/s/mm). In certain embodiments, the tortuous microchannel comprises a plurality of alternating curves having an arc angle between the alternating curves between 30° and 40°.

### BRIEF DESCRIPTION OF THE DRAWINGS

For a detailed description of exemplary embodiments of the invention, reference will now be made to the accompanying drawings in which:

FIG. 1 is a schematic representation of an embodiment of a system for evaluating an ability of a blood sample to coagulate;

FIG. 2 is a top view of an embodiment of a biomimetic microfluidic device of the system of FIG. 1;

FIG. 3 is a schematic representation of an embodiment of a computer system;

FIG. 4 is a flowchart of an embodiment of a method for evaluating an ability of a blood sample to coagulate;

FIGS. 5, 6 are graphs representing embodiments of microchannels;

FIG. 7 is a computational fluid dynamic (CFD) simulation of one of the microchannels of FIGS. 5, 6;

FIG. 8 is a graph illustrating maximum wall shear stress as a function of tortuosity index;

FIG. 9 is a graph illustrating wall shear stress deviation as a function of tortuosity index;

FIGS. 10, 11 are CFD simulations of a microchannel network;

FIG. 11 is a graph illustrating wall shear stress as a function of distance through a microchannel network at varying inlet velocity;

FIGS. 13-15 are fluorescence imaging of a microchannel network;

FIG. 16 is a graph illustrating fibrin area coverage in a microchannel network as a function of Heparin concentration;

FIG. 17 is fluorescence imaging of a microchannel network;

FIG. 18 is a graph illustrating fibrin area coverage in a microchannel network as a function of Bivalirudin concentration;

FIG. 19 is a graph illustrating blood flow pressure in a microchannel network at varying Heparin concentrations as a function of time;

FIG. 20 is a graph illustrating exemplary clotting times at different Heparin concentrations;

FIG. 21 is a graph illustrating exemplary blood flow pressures in a microchannel network at varying Bivalirudin concentrations as a function of time;

FIG. 22 is a graph illustrating exemplary clotting times at different Bivalirudin concentrations;

FIG. 23 is a graph illustrating exemplary blood flow pressures in a microchannel network at varying platelet counts as a function of time;

FIG. 24 is a graph illustrating exemplary clotting times at different platelet counts;

FIG. 25 is a graph illustrating exemplary blood flow pressures in a microchannel network at varying tranexamic acid concentrations as a function of time;

FIG. 26 is a graph illustrating exemplary clotting times at different tranexamic acid concentrations; and

FIG. 27 is a graph illustrating exemplary blood flow pressures for different patients as a function of time.

### DETAILED DESCRIPTION OF THE PREFERRED EMBODIMENTS

The following discussion is directed to various exemplary embodiments. However, one skilled in the art will understand that the examples disclosed herein have broad application, and that the discussion of any embodiment is meant only to be exemplary of that embodiment, and not intended to suggest that the scope of the disclosure, including the claims, is limited to that embodiment.

Certain terms are used throughout the following description and claims to refer to particular features or components. As one skilled in the art will appreciate, different persons may refer to the same feature or component by different names. This document does not intend to distinguish between components or features that differ in name but not function. The drawing figures are not necessarily to scale. Certain features and components herein may be shown exaggerated in scale or in somewhat schematic form and some details of conventional elements may not be shown in interest of clarity and conciseness.

Unless the context dictates the contrary, all ranges set forth herein should be interpreted as being inclusive of their endpoints, and open-ended ranges should be interpreted to include only commercially practical values. Similarly, all lists of values should be considered as inclusive of intermediate values unless the context indicates the contrary.

In the following discussion and in the claims, the terms “including” and “comprising” are used in an open-ended fashion, and thus should be interpreted to mean “including, but not limited to . . . ” Also, the term “couple” or “couples” is intended to mean either an indirect or direct connection. Thus, if a first device couples to a second device, that connection may be through a direct engagement between the two devices, or through an indirect connection that is established via other devices, components, nodes, and connections. In addition, as used herein, the terms “axial” and “axially” generally mean along or parallel to a particular axis (e.g., central axis of a body or a port), while the terms “radial” and “radially” generally mean perpendicular to a particular axis. For instance, an axial distance refers to a distance measured along or parallel to the axis, and a radial distance means a distance measured perpendicular to the axis. Any reference to up or down in the description and the claims is made for purposes of clarity, with “up”, “upper”, “upwardly”, “uphole”, or “upstream” meaning toward the surface of the borehole and with “down”, “lower”, “downwardly”, “downhole”, or “downstream” meaning toward the terminal end of the borehole, regardless of the borehole orientation. As used herein, the terms “approximately,” “about,” “substantially,” and the like mean within 10% (i.e., plus or minus 10%) of the recited value. Thus, for example, a recited angle of “about 80 degrees” refers to an angle ranging from 72 degrees to 88 degrees.

As described above, several clinical scenarios (ex. surgery, trauma, life support devices etc.) require anticoagulation or platelet therapy and consequently, manage their serious consequences of bleeding or thrombosis. Conventional coagulation and platelet function tests, such as, for example, ACT, aPTT, thromboelastography, and platelet aggregometry, are often imprecise, resulting in false positives and false negatives. This aspect limits their ability to predict thrombotic status in clinical settings. For example, despite routine hemostasis monitoring via conventional testing bleeding still occurs in approximately 70% of pediatric patients on ECMO and is independently associated with higher daily risk of mortality. This high bleeding occurrence is partly due to the fact that the conventional coagulation tests (activated clotting time, activated partial thromboplastin time, Anti-Xa) used for unfractionated heparin monitoring lack specificity and sensitivity and often result in false positive or negative cases in the clinic, thus providing misleading information about a patient's coagulation status. Also, the results produced by these assays can vary considerably depending on sample preparation, addition of activators, equipment, and user expertise. Moreover, these conventional tests do not provide information on platelet function, further limiting their ability to predict bleeding and thrombotic risk.

An underlying contributor to the inaccuracies with conventional tests is that conventional tests generally fail to incorporate the mechanical and biochemical cues that activate clotting in vivo. Particularly, whole blood thrombosis is highly dependent on hemodynamic forces (e.g., flow and shear stresses) and cellular interactions. For instance, flow acceleration and deceleration, resulting in fluid shear gradients, have may initiate platelet aggregation during arterial thrombosis in vivo, and clotting in extracorporeal life support devices usually occurs at sites of sudden flow disturbances, stagnation points and stenosed sections of tubing.

In addition to the conventional tests described above, limited microfluidic assays may be utilized which attempt to mimic a parallel network of stenosed arteriole vessels such that whole human blood may be exposed to pathophysiological shear rates and gradients. While such conventional microfluidic assay may more reliably predict thrombosis when compared to conventional laboratory tests, conventional assays typically require more than 1 milliliter (mL) of human blood and, in some cases, requires more than 20 minutes to complete analysis of the human blood tested by the assay. Therefore, despite having some biomimetic qualities, conventional microfluidic assays may not be well suited for rapid analysis where minimal amount of blood use is available, such as, for example, pediatric applications.

Tortuous blood vessels have been shown to induce fluid dynamical disturbances and shear gradients that make them hotspots for forming thrombi in vivo. Additionally, increased thrombosis may develop due to tortuosity in vitro. While conventional microfluidic assays may have some biomimetic qualities as described above, conventional microfluidic assays do not mimic tortuous blood vessels. Conversely, embodiments disclosed herein harness these biological architectural principles and provide a biomimetic system for evaluating an ability of a blood sample to coagulate and an associated biomimetic microfluidic device which integrates shear-gradients caused by tortuosity to stenosis-like expansion contractions in the microfluidic format. Embodiments of biomimetic systems disclosed herein thereby achieve clotting time within only a few minutes and consumes approximately 500 microliters (μL) of human blood, significantly less than conventional microfluidic assays that measure hemostasis or thrombosis. The rapid clotting time achieved from such a minimal sample of blood is achieved via a biomimetic microfluidic device including a tortuous microchannel configured to maximize shear stresses and shear gradients applied to a blood flow therethrough at a given flowrate. Furthermore, embodiments disclosed herein of systems providing tortuosity-activated assays may be of use in thrombin inhibitor dose monitoring, evaluating platelet count, antifibrinolytics, and/or other purposes. Further, embodiments of biomimetic systems disclosed herein may be used to assess blood samples of pediatric patients in critical care who were on extracorporeal membrane oxygenation receiving anticoagulant therapy.

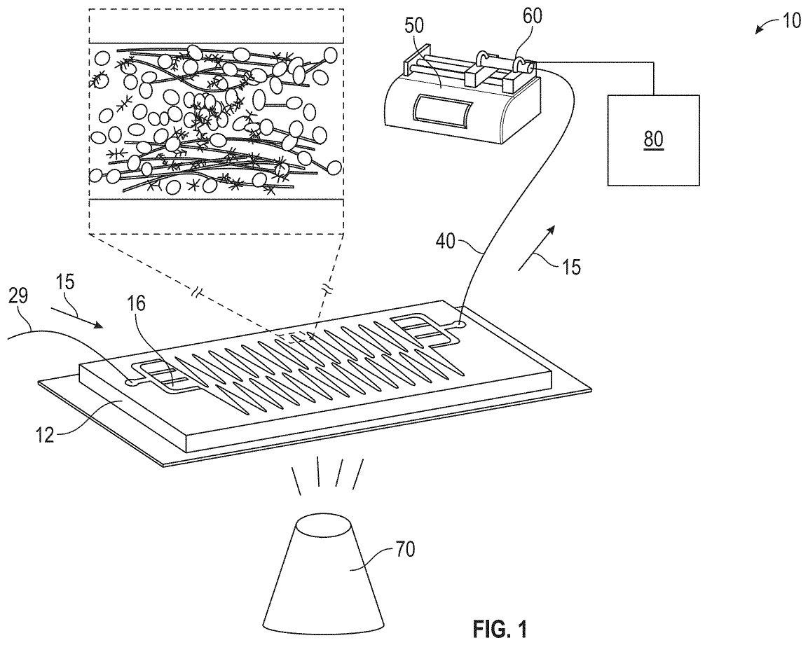

Referring initially to FIG. 1, an embodiment of a system 10 for evaluating an ability of a blood sample to coagulate and/or clot. As will be described further herein, system 10 may be used for coagulating and/or clotting a blood flow via a microfluidic device which mimics tortuous blood vessel networks is shown. As will be described further herein, system 10 is generally configured to effect whole blood occlusion of less than 1-5 ml of human blood in less than approximately 10-20 minutes. In this exemplary embodiment, system 10 generally includes a biomimetic microfluidic device 12, a pump 50 fluidically connected to the biomimetic microfluidic device 12, a sensor 60, an imaging device or microscope 70, and a controller 80 which may be in signal communication with the pump 50, sensor 60, and/or microscope 70.

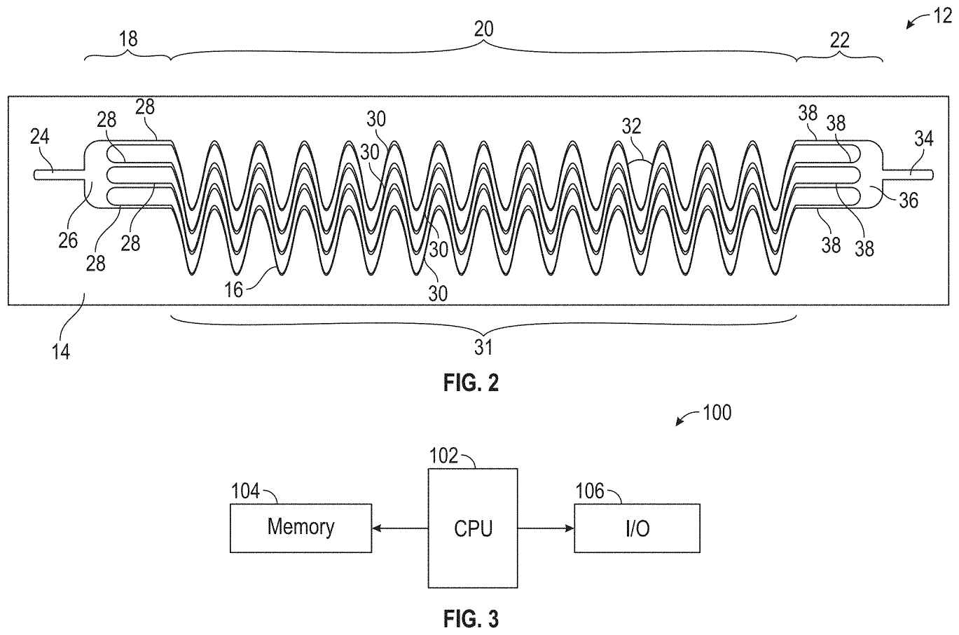

The microfluidic device 12 of system 10 is generally configured to mimic stenosed tortuous arterioles of the human body to create sudden fluid acceleration, tortuosity and non-uniform shear stresses, and sudden deceleration. Referring to FIGS. 1, 2, a top view of the microfluidic device 12 of system 10 is shown. Microfluidic device 12 generally comprises a body or substrate 14 in which a microchannel network 16 is formed. In this exemplary embodiment, body 14 of microfluidic device 12 comprises cured polydimethylsiloxane (PDMS); however, in other embodiments, body 14 of microfluidic device 12 may comprise various materials including polymeric and non-polymeric materials. The body 14 may have a length and a width sized to fit on a standard glass microscope slide.

In this exemplary embodiment, microchannel network 16 comprises an upstream pre-stenosed region 18, a tortuous region 20, and a downstream post-stenosed region 22 where tortuous region 20 is positioned between the pre-stenosed region 18 and the post-stenosed region 22. In other embodiments, microchannel network 16 may only include tortuous region and may not include pre-stoned region 18 and/or post-stenosed region 22. Pre-stenosed region 18 of microchannel network 16 is configured to induce an abrupt acceleration in a fluid or blood flow (indicated by arrows 15 in FIG. 1) through the pre-stenosed region 18 of microchannel network 16. In this exemplary embodiment, pre-stenosed region 18 comprises a fluid inlet or port 24, an inlet fluid reservoir 26, and a plurality of rectilinearly extending inlet fluid microchannels 28 having a rectangular cross-section and extending downstream from the inlet fluid reservoir 26. Fluid inlet 24 of pre-stenosed region 18 may fluidically connect to an inlet fluid conduit or tubing 29 (shown in FIG. 1) extending from microfluidic device 12. As will be described further herein, human blood may be supplied to the microfluidic device 12 via the inlet tubing 29 connected to pre-stenosed region 18 of microchannel network 16.

A flow area of inlet fluid reservoir 26 is substantially greater than a flow area of each of the inlet fluid microchannels 28 extending therefrom, causing blood flow 15 to suddenly accelerate as the fluid is divided between and enters inlet fluid microchannels 28. For example, inlet fluid reservoir 26 may have a width approximately between 3 millimeters (mm) and 6 mm while each of the four inlet fluid microchannels 28 may have a width of approximately between 150 μm and 250 μm; however, in other embodiments, the width of inlet fluid reservoir 26 and inlet fluid microchannels 28 may vary. The divergence of the fluid flow into a plurality of separate inlet fluid microchannels 28 may simulate a vessel network in vivo in which clots form or detach locally, but pressure still increases due to systemic thrombosis. While in this embodiment, pre-stenosed region 18 includes four inlet fluid microchannels 28, in other embodiments, the number of inlet fluid microchannels 28 may vary. For instance, in some embodiments, pre-stenosed region 18 may include a single inlet fluid microchannel 28 having a substantially smaller flow area than inlet fluid reservoir 26.

In this exemplary embodiment, tortuous region 20 of microchannel 16 comprises a plurality (four) tortuous microchannels 30 each having a rectangular cross-section and extending from one of the corresponding inlet fluid microchannels 28 of pre-stenosed region 18. In some embodiments, each tortuous microchannel 30 may have a width of approximately between 150 μm and 250 μm; a height of approximately between 50 μm and 100 μm, and a length of approximately between 25 mm and 150 mm such that each tortuous microchannel 30 may mimic the size of a typical human arteriole of an equivalent diameter (e.g., approximately 100 μm). The cross-sectional shape and/or size of each tortuous microchannels 30 may be similar or equal to the cross-sectional shapes and/or sizes of inlet fluid microchannels 28.

Instead of extending rectilinearly, tortuous microchannels 30 each extend tortuously along alternating bends between pre-stenosed region 18 and post-stenosed region 22. In this exemplary embodiment, tortuous microchannels 30 each comprise a sinusoidal curve corresponding to a given tortuosity index (TI) defined herein as the ratio of the arc length of the tortuous microchannel 30 divided by the end-to-end length 31 of the tortuous microchannel 30. In other embodiments, tortuous microchannels 30 may comprise toroidal curves, steps, waveforms, and/or a mixture of diverse curves derivable from blood vessel imagery. Not intending to be bound by any particular theory, the TI of a given tortuous microchannel 30 may be expressed in accordance with Equation (1) below, where S represents arc length, L represents end-to-end or rectilinear distance between opposing ends of the tortuous microchannel 30, A represents amplitude, a represents frequency, and x represents end-to-end length:

$$TI = \frac{S}{L} = \frac{1}{L} {\int }_{0}^{L} ( \sqrt{1 + {A}^{2} {{α}^{2} ( cos a x )}^{2}} ) d x \qquad ( 1 )$$

In this exemplary embodiment, each tortuous microchannel 30 has a TI of at least 2.0. In some embodiments, each tortuous microchannel 30 has a TI of approximately between 3.0 and 3.4; however, in other embodiments, the TI of each tortuous microchannel 30 may vary. In some embodiments, each tortuous microchannel 30 has a TI of approximately 3.4 and an arc angle 32 of approximately 30° which may correspond to a maximum TI providable by tortuous microchannels 30. TI=3.4 is a design constraint for this configuration, but in other configurations, TI may have a different working range. A maximum wall shear stress in the blood flow 15 through tortuous microchannels 30 at a given fluid flowrate may be positively correlated with the TI of the tortuous microchannels 30. Thus, by maximizing the TI of tortuous microchannels 30 the maximum wall shear stress and/or a maximum shear rate/shear rate gradient in the fluid flow through tortuous microchannels 30 at a given fluid may in-turn be maximized. Additionally, shear gradients (acceleration and deceleration of blood flow 15) across each tortuous microchannel 30 also increase with increasing TI and thus shear gradients may be maximized by maximizing the TI of each tortuous microchannel 30. Given that occlusion of blood flow 15 may occur more rapidly a given fluid flowrate in response to increasing wall shear stress and shear gradients of blood flow 15, the time required of occlusion of blood flow 15 to occur at a given fluid flowrate may be minimized by maximizing the TI of each tortuous microchannels 30. In this manner, by providing each tortuous microchannel 30 with the maximum allowable TI of 3.4, tortuous microchannels 30 are configured to provide a highly pathological and prothrombotic fluid mechanical environment to minimize the time required for forming blood clots and occluding blood flow 15 at a given fluid flowrate.

In this exemplary embodiment, post-stenosed region 22 comprises a fluid outlet or port 34, an outlet fluid reservoir 36, and a plurality of rectilinearly extending outlet fluid microchannels 38 having a rectangular cross-section and extending downstream from the plurality of tortuous microchannels 30 but upstream from outlet fluid reservoir 36. Fluid outlet 34 of post-stenosed region 22 may fluidically connect to an outlet fluid conduit or tubing 40 (shown in FIG. 1) extending from microfluidic device 12. Outlet fluid conduit 40 is connected to the pump 50 and sensor 60 of system 10 as will be described further herein. Similar to pre-stenosed region 18, a flow area of outlet fluid reservoir 36 is substantially greater than a flow area of each of the outlet fluid microchannels 38 positioned upstream from outlet fluid reservoir, causing blood flow 15 to suddenly decelerate as the blood flows into the outlet fluid reservoir 36. In some embodiments, the size (e.g., width, height, and/or length) and/or shape of outlet fluid reservoir 36 may be similar to the size and/or shape of inlet fluid reservoir 26 while the size and/or shape of each outlet fluid microchannel 38 may be similar to the size and/or shape of each inlet fluid microchannel 28; however, in other embodiments, the size and/or shape of outlet fluid reservoir 36 may vary from the size and/or shape of inlet fluid reservoir 26, and the size and/or shape of each outlet fluid microchannel 38 may vary from the size and/or shape of each inlet fluid microchannel 28. The deceleration of the blood flow 15 as it enters outlet fluid reservoir 36 promotes shear gradients in the blood flow 15 to enhance clotting of the blood and occlusion of blood flow 15.

Referring still to FIGS. 1, 2, pump 50 of system 10 is connected to the outlet fluid conduit 40 and is generally configured to draw blood 15 through the microchannel network 16 of microfluidic device 12 from fluid inlet 24 to fluid outlet 34 at either a fixed or constant flowrate over time or at a fixed or constant fluid pressure over time. In some embodiments, pump 50 comprises a syringe pump; however, in other embodiments, the configuration of pump 50 may vary. In some embodiments, pump 50 is configured to pump blood flow 15 through microchannel network 16 at a constant flowrate of approximately between 40 microliters per minute (μl/min) and 100 μl/min. In certain embodiments, pump 50 is configured to pump blood flow 15 through microchannel network 16 at a constant flowrate of approximately 70 μl/min. In some embodiments, an inner surface defining microchannel network 16 may be coated with a thrombus forming agent, a reagent, collagen, and a thrombus inhibiting agent, a platelet activating material, a platelet inhibiting material, a fibrin network forming material, a fibrin network disrupting material, and/or cells.

While increasing shear stresses and shear gradients in blood flow 15 (minimizing the time required for blood clotting) may be achieved by increasing the flowrate provided by pump 50, increased flowrates also result in an increase in the consumption of blood by system 10. A flowrate of approximately 70 μl/min provided by pump 50 may advantageously achieve clotting and occlusion of the blood flow 15 within only a few minutes while only consuming approximately 500 μl of blood. Consuming less than one ml of blood may allow system 10 to be utilized in applications in which only a minimal amount of blood may be consumed such as, for example, pediatric applications.

Sensor 60 of system 10 is configured to measure or determine pressure and/or flowrate of the blood flow 15 flowing through the microchannel network 16 of microfluidic device 12. Sensor 60 may be connected to or otherwise in signal communication with controller 80 which may display (e.g., via a visual display of the device 80) and/or record the flowrate and/or pressure measurements made by the sensor 60. While in this embodiment sensor 60 is coupled between the outlet fluid conduit 40 and the pump 50, in other embodiments, sensor 60 may be positioned in other locations.

As clotting begins to occur and blood flow 15 becomes occluded, the resistance to the flow of blood flow 15 through the microchannel network 16 of microfluidic device 12 increases. In an embodiment, pump 50 may provide blood flow 15 with a constant flowrate through microchannel network 16 while sensor 60 measures pressure of blood flow 15. By detecting an increase in pressure of blood flow 15 at the constant flowrate provided by pump 50, sensor 60 may in real-time register or detect the formation of thrombi within blood flow 15. Similarly, in embodiments where pump 50 provides a constant pressure over time at a varying flowrate, sensor 60 may in real-time register or detect the formation of thrombi within blood flow 15 by detecting a reduction in the flowrate of blood flow 16 at the constant pressure provided by pump 50.

Imaging device 70 of system 10 may allow for the qualitative and visual detection of thrombi formation and occlusion of blood flow 15 within the microchannel network 16 of microfluidic device 12. For example, imaging device 70 may be used to observe fluorescently-labeled fibrinogen (e.g., approximately 15 micrograms per milliliter (μm/ml), Alexa Fluor 647, Invitrogen, Gran Island, N.Y.) and/or platelets (e.g., 10 microliters per milliliter (μl/ml) Human CD41-PE (clone:IPL3), Invitrogen) added to the blood flow 15 via time-lapse imaging by imaging device 70 of the microchannel network 16. In some embodiments, imaging device 70 may provide a 10×, NA 0.3 objective; however, in other embodiments, the configuration of imaging device 70 may vary. In other embodiments, system 10 may not include imaging device 70 and instead may only include sensor 60 for detecting thrombi formation and the occlusion of blood flow 15.

The controller 80 of system 10 is generally configured to control the operation of pump 50 and to quantitatively detect the coagulation of blood flow 15 based on measurements of pressure and/or flowrate of blood flow 15 provided by sensor 60. As will be discussed further herein, controller 80 may comprise a computer system including one or more processors and memory devices.

Controller 80 may determine the coagulation of blood flow 15 by monitoring in real-time an increase in a resistance to the flow of blood flow 15 over time. In some embodiments, controller 80 may determine a first or initial pressure of blood flow 15 at an initiation of the flow of blood flow 15 caused by the activation pump 50, and a time (e.g. determined via a timer of controller 80) at which the pressure of blood flow 15 is approximately between 2.3 times and 3.0 times greater than the initial pressure of blood flow 15. In some embodiments, controller 80 determines the time at which the pressure of blood flow 15 is approximately between 2.3 times and 3.0 times greater than the initial pressure of blood flow 15 as the clotting time of the blood perfused through the microfluidic device 12 of system 10 and which corresponds to the achievement of hemostasis of the blood perfused through microfluidic device 12. In some embodiments, the 2.3 fold to 3.0 fold in pressure increase of blood flow 15 used to determine clotting time is based on a phenomenological mathematical model to quantify the time at which microchannel network 16 is occluded with thrombus. The mathematical model may be based on the observation that fibrin formation follows a sigmoidal trend, allowing for the establishment of a two-parameter exponential growth curve (reciprocal of sigmoid) to fit the mathematical model. In other embodiments, controller 80 may determine the time at which the flowrate of blood flow 15 is approximately between 2.3 times and 3.0 times less than an initial flowrate of blood flow 15 as the clotting time of the blood perfused through the microfluidic device 12 of system 10 and which corresponds to the achievement of hemostasis of the blood perfused through microfluidic device 12.

The system 10 and associated microfluidic device 12 shown in FIGS. 1, 2 may be utilized in a variety of both pharmaceutical and clinical applications for evaluating an ability of a blood sample to coagulate. For instance, system 10 may be utilized to detect changes in clotting time due to platelet count under stenosed and tortuous blood flow 15. System 10 may be used to predict conditions associated with platelet count such as, for example, platelet transfusion. Additionally, system 10 could be used to monitor antifibrinolytic therapy in practical settings where hyperfibrinolysis may arise. Further, system 10 may be used to measure defects in hemostasis present in a blood sample. For instance, the rapidity at which system 10 may determine a clotting time from a minute blood sample enables system 10 to be utilized in detecting defects in hemostasis in a blood sample from a pediatric patient in critical care with altered hemostasis due to, for example, the effects of anticoagulation and/or acquired symptoms due to operating procedures.

Referring now to FIG. 3, an embodiment of a computer system 100 suitable for implementing one or more embodiments disclosed herein is shown. For example, components of system 10 shown in FIGS. 1, 2 (e.g., controller 80, etc.) may be configured in a manner similar to the computer system 100 shown in FIG. 3. The computer system 100 includes a processor 102 (which may be referred to as a central processor unit or CPU) that is in communication with one or more memory devices 104, and input/output (I/O) devices 106. The processor 102 may be implemented as one or more CPU chips. The memory devices 104 of computer system 100 may include secondary storage (e.g., one or more disk drives, etc.), a non-volatile memory device such as read only memory (ROM), and a volatile memory device such as random access memory (RAM). In some contexts, the secondary storage ROM 106, and/or RAM comprising the memory devices 104 of computer system 100 may be referred to as a non-transitory computer readable medium or a computer readable storage media. I/O devices 106 may include printers, video monitors, liquid crystal displays (LCDs), touch screen displays, keyboards, keypads, switches, dials, mice, and/or other well-known input devices.

It is understood that by programming and/or loading executable instructions onto the computer system 100, at least one of the CPU 102, the memory devices 104 are changed, transforming the computer system 100 in part into a particular machine or apparatus having the novel functionality taught by the present disclosure. Additionally, after the computer system 100 is turned on or booted, the CPU 102 may execute a computer program or application. For example, the CPU 102 may execute software or firmware stored in the memory devices 104. During execution, an application may load instructions into the CPU 102, for example load some of the instructions of the application into a cache of the CPU 102. In some contexts, an application that is executed may be said to configure the CPU 102 to do something, e.g., to configure the CPU 102 to perform the function or functions promoted by the subject application. When the CPU 102 is configured in this way by the application, the CPU 102 becomes a specific purpose computer or a specific purpose machine.

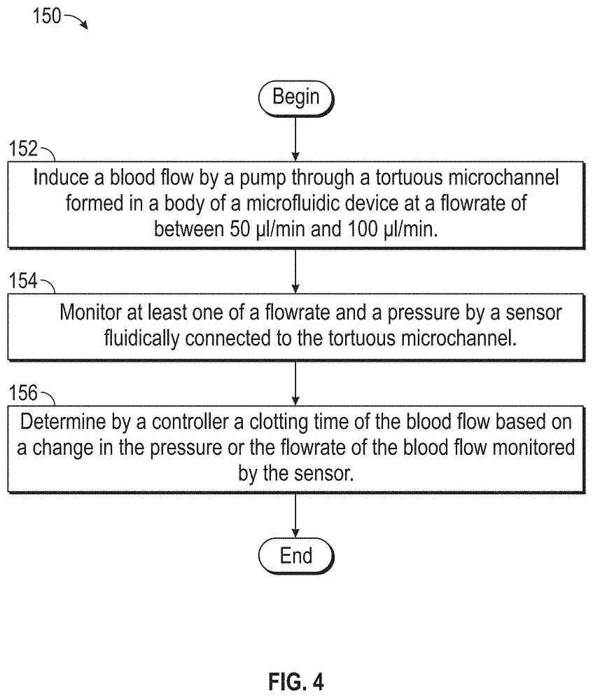

Referring to FIG. 4, an embodiment of a method 150 for coagulating a blood flow is shown. Initially, at block 152 method 150 comprises inducing a blood flow by a pump through a tortuous microchannel formed in a body of a microfluidic device at a flowrate of between 50 μl/min and 100 μl/min. In some embodiments, block 152 comprises inducing the blood flow at a constant flowrate of approximately 70 μl/min. In some embodiments, a maximum shear stress of at least 5 pascals (Pa) is applied to the blood flow in the tortuous microchannel. In some embodiments, a shear gradient of greater than 5000 per second per millimeter (1/s/mm) is achieved in the blood flow in the tortuous microchannel. For example, in some embodiments a shear gradient of approximately between 5000 1/s/mm and 15000 1/s/mm may be achieved. In some embodiments, block 152 comprises inducing the blood flow 15 by the pump 50 through the tortuous microchannels 30 of the microfluidic device 12 shown in FIGS. 1, 2.

At block 154, method 150 comprises monitoring at least one of a flowrate and a pressure by a sensor fluidically connected to the tortuous microchannel. In some embodiments, block 154 comprises monitoring at least one of a flowrate and a pressure of the blood flow 15 by the sensor 60 of system shown in FIG. 1. For example, block 154 may comprise monitoring a variable pressure of blood flow 15 as pump induces the blood flow 15 at a constant pressure. Alternatively, block 154 may comprise monitoring a variable flowrate of blood flow 15 as pump induces the blood flow 15 at a constant flowrate.

At block 156, method 150 comprises determining by a controller a clotting time of the blood flow based on a change in the pressure or the flowrate of the blood flow monitored by the sensor. In some embodiments, block 156 comprises determining by controller 80 a clotting time of the blood flow 15 based on a change in the pressure or the flowrate of the blood flow 15 monitored by sensor 60 of the system 10 shown in FIG. 1. For example, a clotting time may correspond to a time period at which the pressure of the blood flow 15 is between 2.3 times and 3.0 times greater than an initial pressure of the blood flow 15 at an initiation of the blood flow induced by the pump 50.

Experimental testing was conducted to develop a prototype or experimental system for clotting blood having features in common with the system 10 shown in FIGS. 1, 2. The system and experimental microfluidic device described below may have features in common with the system 10 microfluidic device 12 shown in FIGS. 1, 2; however, it may be understood that system 10 and microfluidic device 12 described above are not limited by the discussion of the system and experimental microfluidic device described below.

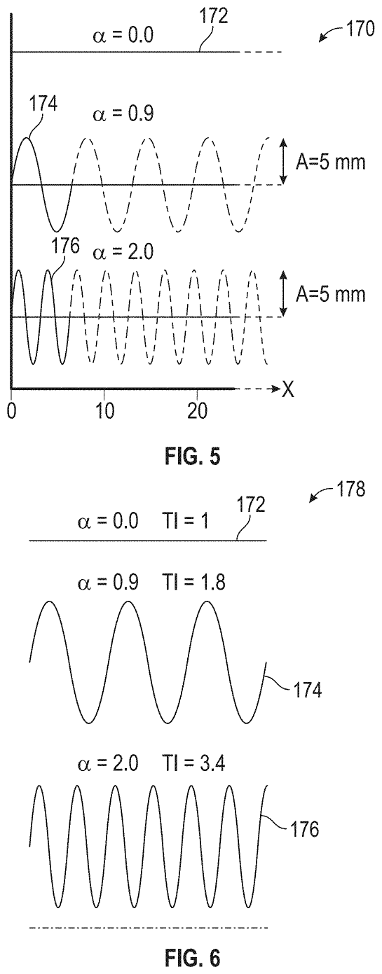

Initially, the criteria required in the design of tortuous microchannels was investigated. Referring to FIGS. 5, 6, a graph 170 is shown including three microchannel designs 172, 174, and 176 having frequencies 0, 0.9, and 2.0 are shown. Microchannel designs 172, 174, and 176 of were initially generated in SolidWorks™ (SolidWorks Corporation, 300 Baker Avenue, Concord, Mass. 01742) using the equation of a sinusoidal curve y=A sin(αx), where A represents amplitude, a represents frequency, and x represents the end-to-end length. The amplitude and end-to-end length were fixed at 5 mm and 56 mm. The primary dimensions—width, height and length—were set to 200 μm, 75 μm and 56 mm, respectively, for each microchannel design 172, 174, and 176 to mimic the size of a typical arteriole of an equivalent diameter ˜100 μm. Also, this configuration was convenient to fabricate, image, perfuse with blood, and fit on a standard glass slide. As shown particularly in the graph 178 of FIG. 6, a commonly used TI for blood vessels was applied to microchannel designs 172, 174, and 176. As described above, TI is defined as the ratio of vessel arc length over the line distance between the two ends of the respective microchannel as expressed in Equation (1) above. Upon varying frequency, microchannels with a TI ranging from unity (straight channel) (microchannel design 172) to a maximum of 3.4 (30° arc angle) (microchannel design 176) could be designed, beyond which the channels could not be further bent. However, this range in TI is typically observed in blood vessels in vivo.

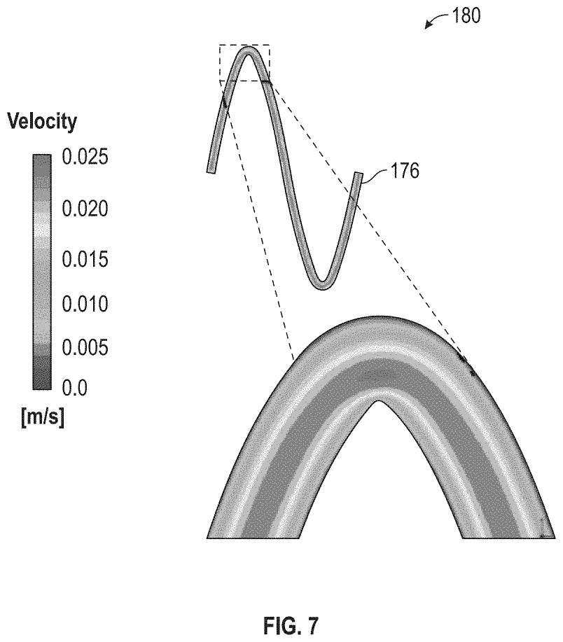

The inherent fluid dynamics in microchannels that met the pertinent design criteria were then investigated with the aim to predict the propensity to enhance blood clotting due to vascular tortuosity. The CAD drawings of tortuous microchannels (e.g., microchannel design 176) were imported to fluid modelling software (ANSYS® software), and CFD simulations were performed of blood flow assuming blood as a complex non-Newtonian fluid. Referring to FIG. 7, a CFD simulation 180 of the microchannel 176 is shown. Through this numerical modelling, the hemodynamic profiles for varying tortuosity indices were determined, which showed maximum velocity at the center and no slip at the boundary as expected from the model.

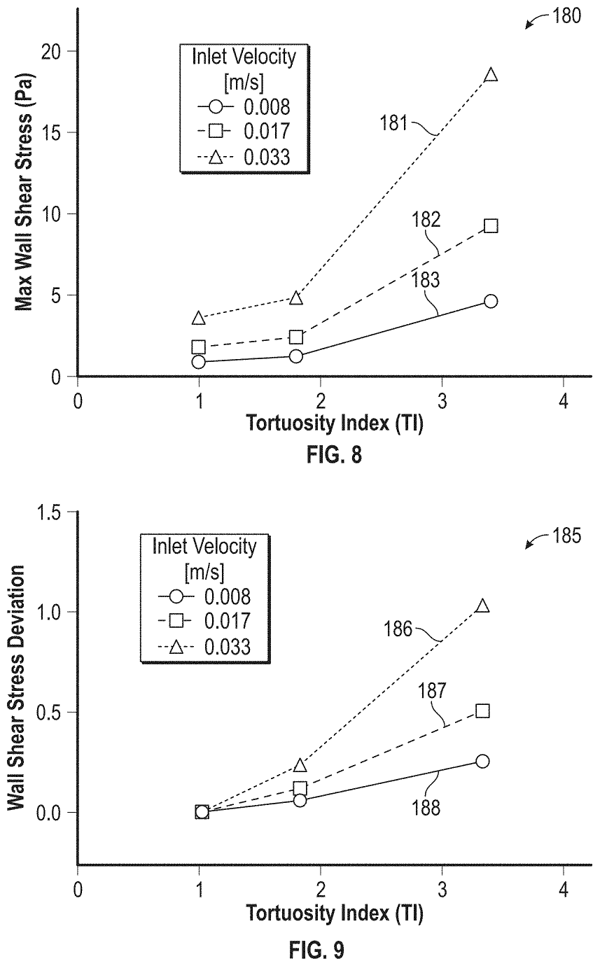

Referring to FIG. 8, a graph 180 of max wall shear stress as a function of TI is shown for three separate inlet velocities 181 (0.008 meters per second (m/s)), 182 (0.017 m/s), and 183 (0.033 m/s). The CFD analyses revealed that the maximum wall shear stress increased with increasing tortuosity and imposed inlet velocity boundary condition, as shown in graph 180. Referring to FIG. 9, a graph 185 of wall shea stress deviation as a function of TI is shown for three separate inlet velocities 186 (0.008 meters per second (m/s)), 187 (0.017 m/s), and 188 (0.033 m/s). Importantly, analysis of deviations from the mean across the channel showed that non-Newtonian flow in tortuous vessels also led to fluid shear gradients (acceleration and deceleration of flow) across the microchannel that also increased with tortuosity, as shown in graph 185. These data thus predict that upon blood perfusion in a tortuous microchannel, the occlusion may be fastest if these channels were most tortuous. Based on this analysis, it was decided to design an experimental microfluidic device consisting channels of a tortuosity index, TI=3.4. Further, since the analysis also showed that shear stresses and gradients increase upon increasing flowrates, more occlusion at high shear was predicted. However, in a single-pass device, a high flowrate may also lead to more blood consumption. Therefore, experimental optimization was required that leads to faster clotting due to high shear while requiring a relatively low blood volume.

An objective of creating the experimental microfluidic device was to design a microfluidic device that integrates the shear gradients induced by stenosis to tortuosity-driven gradients in the microdevice, so that blood clots could also form more rapidly while also requiring lower blood volume. Therefore, the experimental microfluidic device (e.g., a device having features in common with microfluidic device 12 shown in FIGS. 1, 2) was configured to mimic stenosed tortuous arterioles to create sudden fluid acceleration (pre-stenosed) upon blood perfusion via a pre-stenosed region of the device, followed by a region of tortuosity and non-uniform shear (stenosed+tortuous), and then by a region with a sudden deceleration.

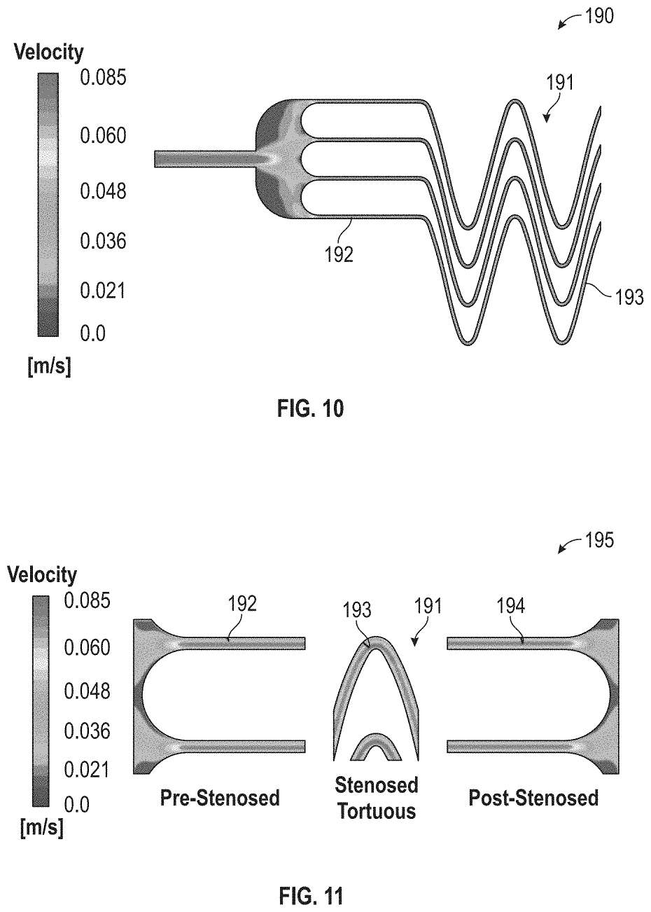

Referring to FIGS. 10, 11, CFD simulations 190, 195, respectively of pre-stenoses 192, stenosed tortuous 193, and post-stenosed 194 regions of a microchannel 191 are shown. CFD simulations 190, 195 confirmed that the fluid undergoes several acceleration and deceleration stages (e.g., regions 192, 193, and 194) along the microfluidic device. Correspondingly, it was found that the wall shear stress rapidly changes at the pre-tortuous and post-tortuous region.

Referring to FIG. 12, a graph 200 of wall shear stress as a function of distance of the microchannel 191 is shown for inlet velocities 201 of 0.039 m/s, 202 of 0.078 m/s, and 203 of 0.117 m/s. By introducing tortuosity, it was shown in graph 200 that shear also fluctuates significantly in the tortuous region 193, creating a highly pathological and prothrombotic fluid mechanical environment within the device. Moreover, it was shown that the absolute wall shear rate as well as its gradients increased with increasing inlet velocity, indicating that blood clot formation may be enhanced at higher flowrates. Thus, the microchannel design comprising three distinct shear gradient zones: pre-stenosed, stenosed+tortuous and post-stenosed together contribute to enhanced blood clotting.

A microfluidic device was then fabricated and mounted onto a microscope for visualization. The microfluidic device was also connected the to a syringe pump to introduce flow therethrough in a configuration similar, in at least some respects, to that of system 10 shown in FIG. 1. Blood first entered into a large reservoir (4.7 mm wide) and then flow split into four smaller parallel stenosed tortuous channels (200 μm wide); followed by convergence of the flow into an outlet similar to the inlet. The 4-channel design is partially analogous to a vessel network in vivo in which clots may form or detach locally, but the pressure still increases due to the systemic thrombosis. In addition, the total width and length of the device were designed to fit on a standard glass microscope slide for practical ease. Each device contains multiple single microchannel sections that were optimized to create maximum tortuosity (alternating 30° bends, corresponding to TI=3.4), to expose flowing whole blood to varying shear rates due to both stenosis and tortuosity, thus promoting rapid clot formation and occlusion. To enable more rapid cell activation and adhesion, the surface of the microchannels were further functionalized within the device with collagen type I, which is a commonly applied platelet agonist. The microfluidic device was operated at a flowrate of 70 μl/min, leading to a pre-stenosed mean wall shear rate of 1,200 reciprocal seconds (1/s) corresponding to typical arteriolar flow, and a wall shear gradient of 935 reciprocal second-millimeters (1/(s-m)) in the straight region following the pre-stenosed reservoir.

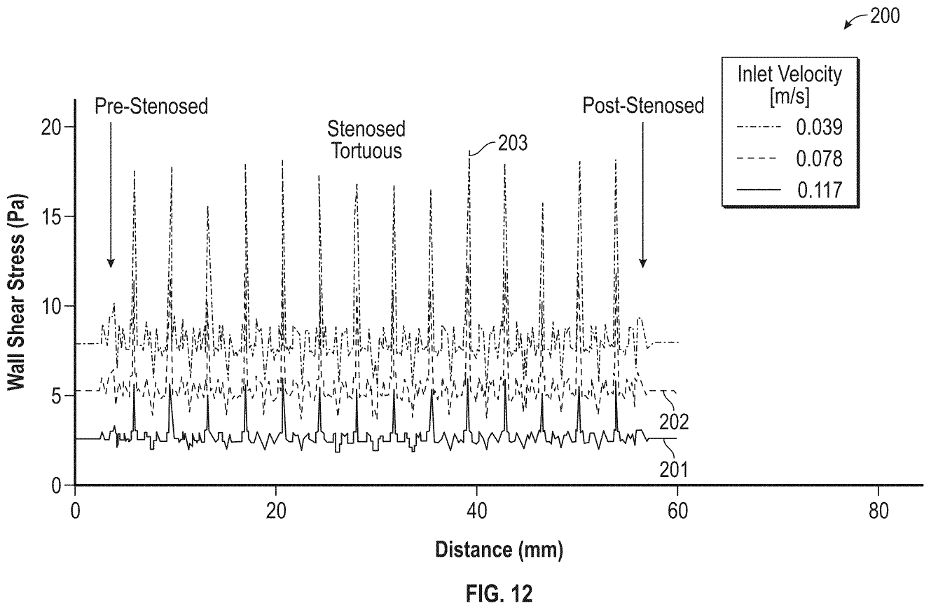

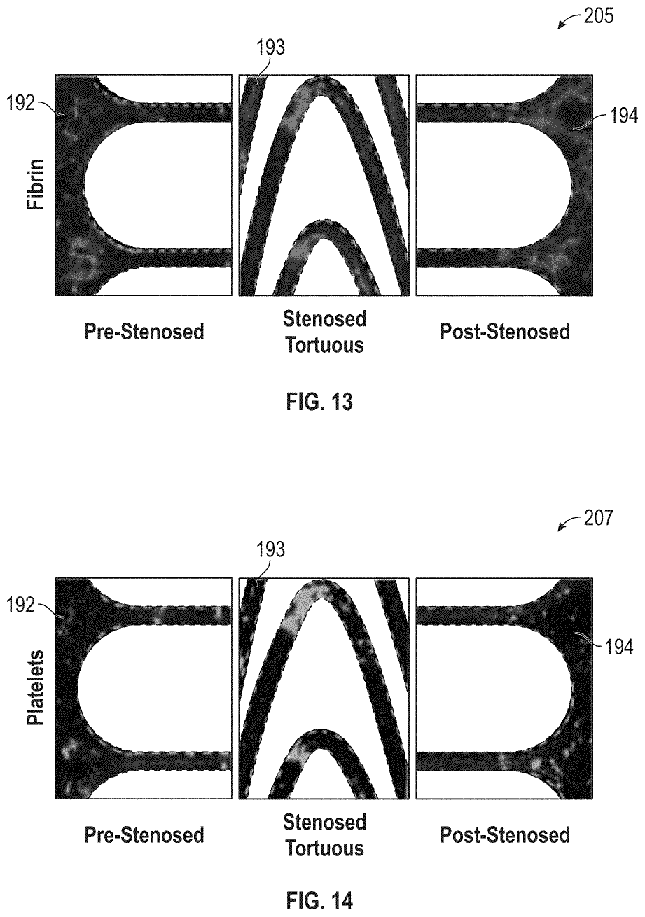

Referring to FIGS. 13, 14, re-calcified citrated whole blood was perfused through the microfluidic device for a maximum of 10 minutes at the 70 μl/min, less than 1 mL of blood was consumed, and the formation of various sized thrombi was detected throughout its entire length using fluorescence imaging 205, 207 shown in FIGS. 13, 14, respectively. This suggested the microfluidic device could provide a tool to measure thrombosis in conditions requiring low blood volume and analysis within a few minutes. Importantly, these in vitro results indicated that physiologically-relevant whole blood thrombus formation may occur inside the microfluidic device underlying a key advantage over standard laboratory tests that do not incorporate flow or require more specific coagulation pathways.

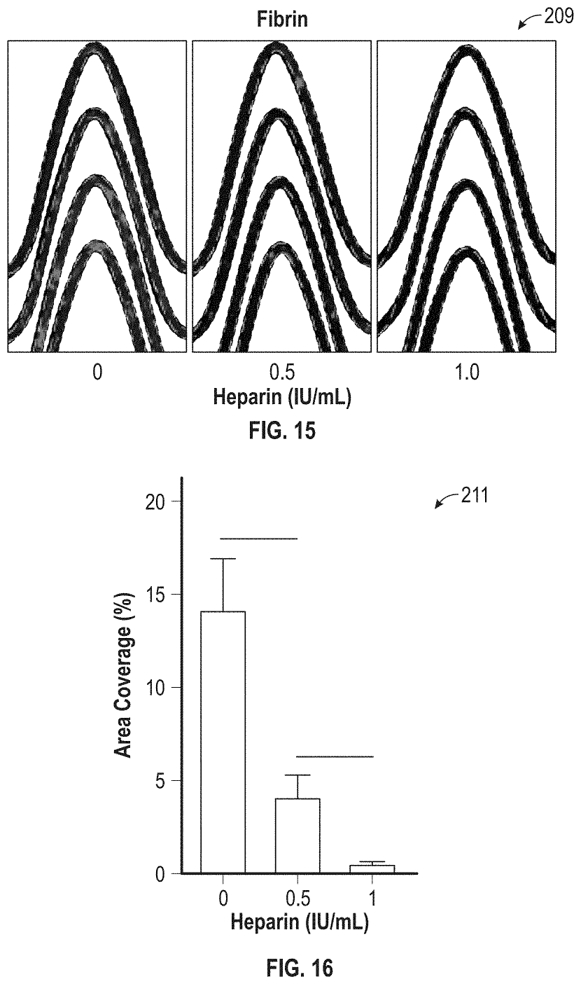

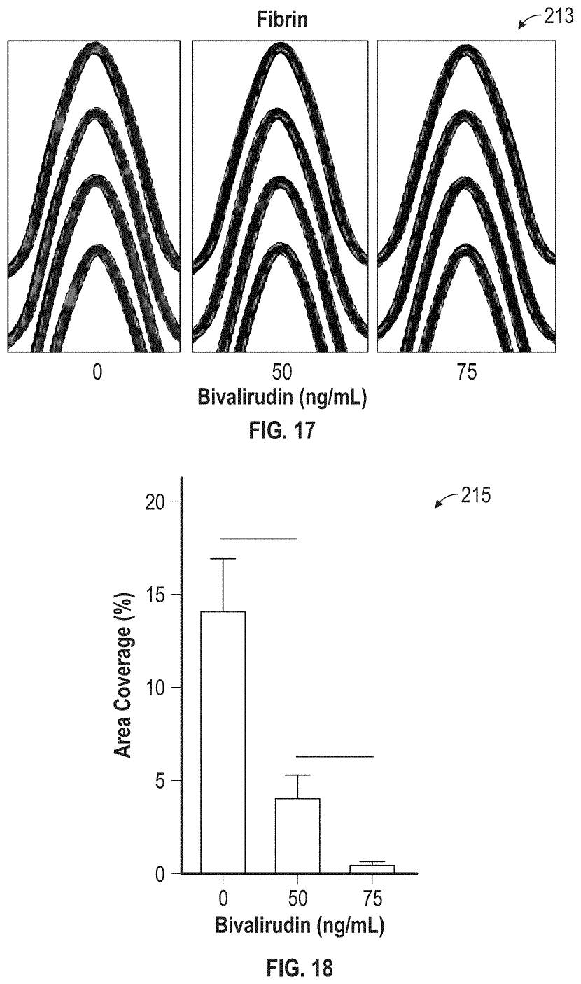

Precise and personalized anticoagulant dose monitoring as close to a real-time basis is critical in patients on extracorporeal assist devices (for example, hemodialysis, membrane oxygenation, mechanical circulatory support, and so on) to ensure therapeutic anticoagulation, and to rapidly detect any life-threatening thrombotic or bleeding events that may arise. To explore the potential utility of using this microfluidic device in monitoring the typical anticoagulants administered in critical care, sensitivity to unfractionated heparin (UFH), an indirect thrombin Inhibitor were tested. Additionally, UFH is the most commonly administered anticoagulant given to patients on extracorporeal assist devices. Referring to FIGS. 15, 16, when clinically-relevant doses of UFH (0-1 international units per milliliter (IU/mL))) were added to whole blood samples fluorescently labelled to track fibrin formation, and the blood was perfused though the device while monitoring fibrin using fluorescence microscopy, decrease in fibrin area coverage as concentration of heparin was increased was found as shown in fluorescent imaging 209 of FIG. 15 and the graph 211 of FIG. 16. These results suggested the microfluidic device can potentially detect differences in heparin dosage within blood samples in-vitro. Next, it was explored if the microfluidic device may also detect differences in doses of bivalirudin, a direct thrombin inhibitor, when added to blood samples. Referring to FIGS. 17, 18, when the same methodology as described above for heparin was followed except that instead clinically relevant doses of bivalirudin 19 (0-100 ng mL−1) was added to whole blood, reduced fibrin was again observed with nearly complete clearance at 75 nanograms per milliliter (ng/ml) demonstrating a potentially unique advantage over current monitoring tools that have limited sensitivity to bivalirudin, as shown in the fluorescent imaging 213 of FIG. 17 and the graph 215 of FIG. 18.

Even though microscopic analysis of real-time thrombus formation in the microfluidic device may be useful in labs, the microfluidic device may also be deployed and utilized at the point-of-care. In the interest of utilizing the microfluidic device at the point-of-care, the microfluidic device was connected to a syringe pump, a disposable pressure sensor, and a display monitor. Given that each of these components are already a part of most extracorporeal circuits, these components may be conveniently integrated in broad critical care settings. Additionally, using these components, clot formation may be automatically tracked in real-time by measuring changes in fluid pressure caused by increasing channel occlusion inside the device, as described above with respect to system 10.

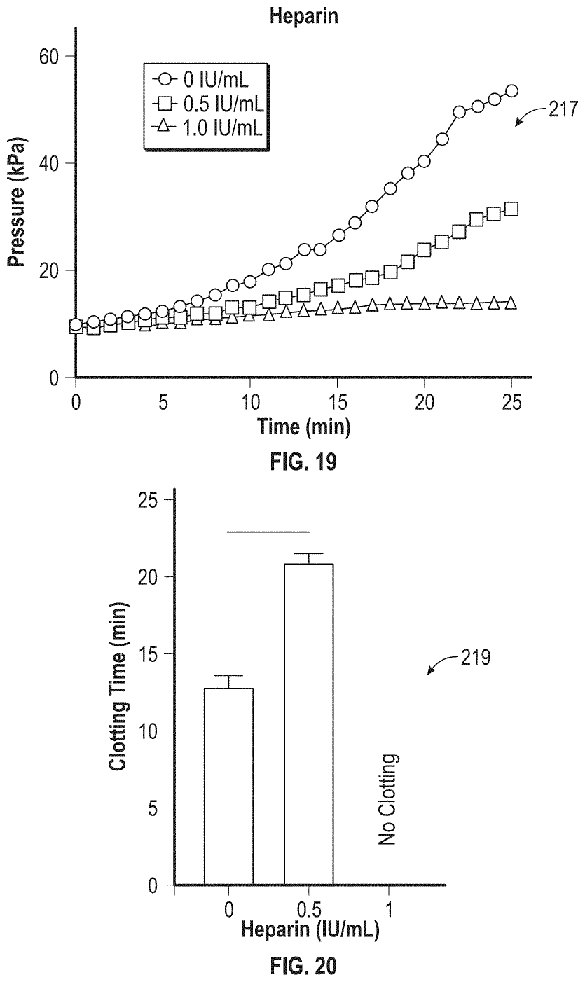

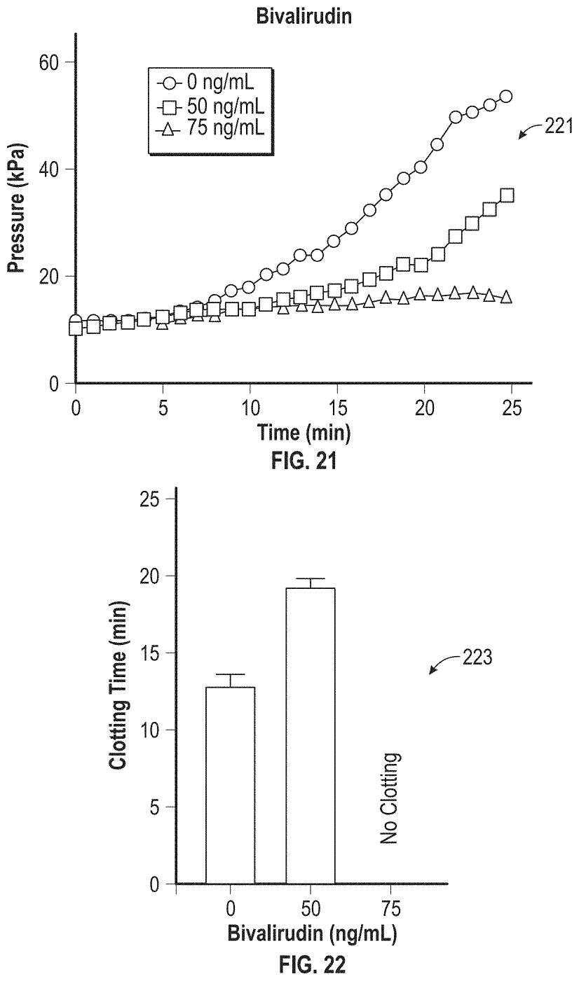

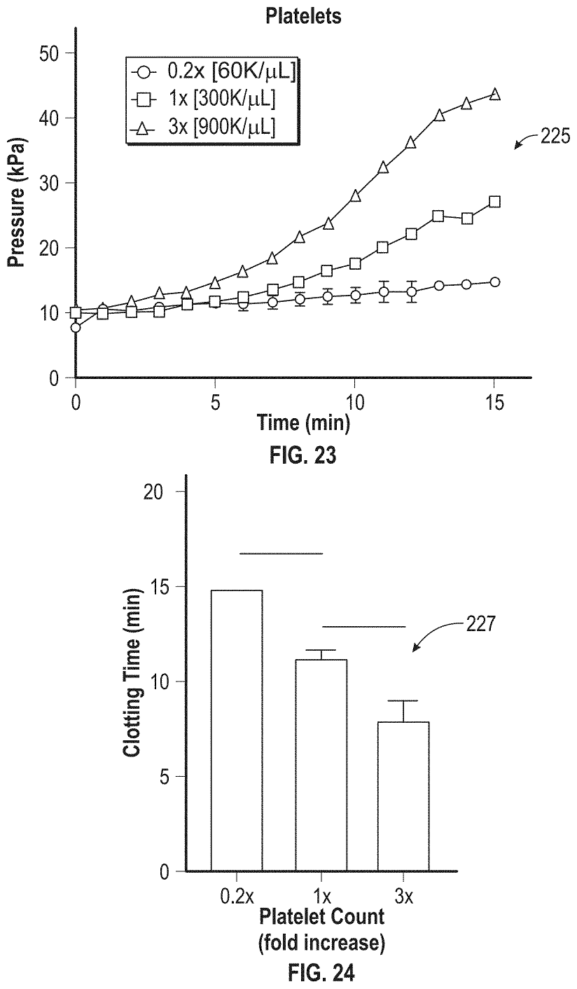

It was found during testing of the microfluidic device that as recalcified citrated whole blood was perfused and formed clots inside the microfluidic device, pressure increases typical of the dynamics of clotting in blood vessel in vivo, or in an in vitro hollow channel, was seen, the pressure increases comprising three stages—a steady reaction time, a growth phase, and saturation. Referring to FIGS. 19, 20, when heparin (0-1 IU/ml) was added into blood, the pressure increase shifted relative to normal controls as heparin concentration was increased, as shown in graphs 217, 219 of FIGS. 19, 20, respectively. Referring to FIGS. 21, 22, a similar trend was identified when we used bivalirudin (0-75 ng/mL) was used, as shown in graphs 221, 223 of FIGS. 21, 22, respectively, demonstrating that this pressure sensor-based setup can detect effect of thrombin inhibitors in-vitro and may potentially be used to monitor them. Next, a clotting time measurement was developed for these pressure readouts to serve as a quantitative end point in lab and clinical settings. Referring to FIGS. 23, 24, a 2.5× pressure increase from baseline measurement was set as an endpoint for determining clotting time, and it was found that clotting time decreased as concentration increased for both heparin and bivalirudin as shown particularly in graphs 219 and 223 of FIGS. 20, 22. Channel occlusion occurred within 13 minutes using less than 1 ml of whole blood in untreated samples, an approximately 35% improvement over conventional microfluidic assays, which indicates that the introduction of tortuosity-driven shear gradients substantially accelerated clot formation.

As platelet count can be a major contributor to the development of vascular occlusion in many clinically settings, it was further explored if the microfluidic device can be used to detect changes in clotting time due to platelet count under stenosed and tortuous flow. First, we measured platelet adhesivity was measured in a microfluidic device coated with collagen (type I, rat, 100 micrograms per milliliter (μg/ml)) using recalcified whole blood with varying platelet counts to explore if the microfluidic device can potentially be used to predict disorders where platelet count is low or elevated. Referring to FIG. 23, 24 blood samples with 0.2× and 3× platelet counts relative to normal controls (1×) were produced and when these samples were perfused, a relative shift of pressure curves to the right and left for the 0.2× and 3× samples was observed, as shown in graph 225 of FIG. 23. Also, when the pressure traces illustrated in graph 225 were measured for clotting time, a dose-dependent decrease in clotting time was observed as a function of increasing platelet count, as shown particularly in graph 227 of FIG. 24. The results illustrated in graphs 225, 227 validate that platelets are a critical component of occlusion within this device since their removal and addition to blood affected clotting dynamics and time. The results also provide a proof-of-concept that the microfluidic device could potentially be used for predicting conditions associated with platelet count or therapy such as, for example, platelet transfusion.

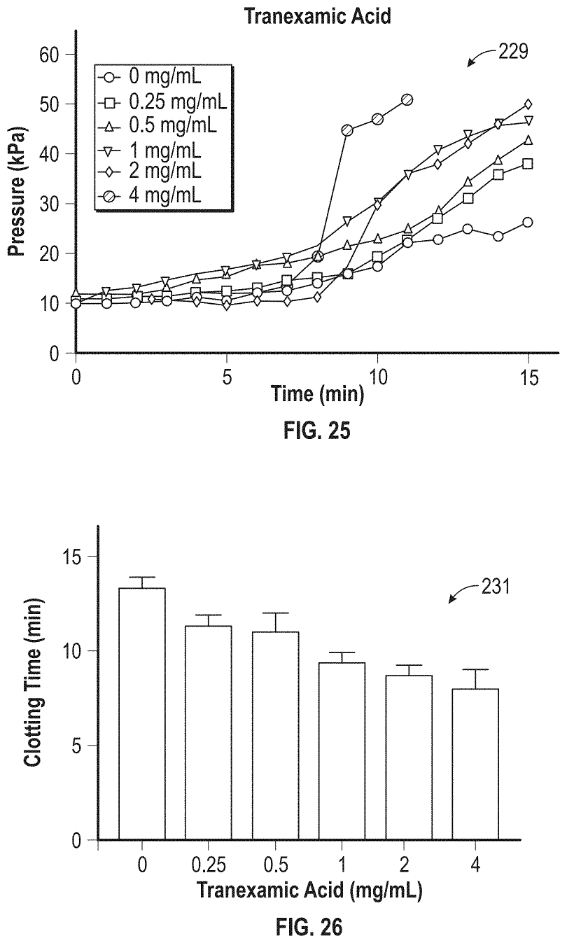

Patients on extracorporeal mechanical systems can experience hyperfibrinolysis and as a result, are sometimes administered antifibrinolytics to decrease the risk of having a bleeding episode. However, the use of antifibrinolytics is debatable partly because there are no assays that can provide reliable diagnostics of impact of delivering these drugs to patients. The sensitivity of the microfluidic device to clinically-relevant doses of tranexamic acid (Cyklokapron™, 0-4 mg/mi) was tested to explore the potential utility of the microfluidic device for monitoring antifibrinolytic therapy. Tranexamic acid is a synthetic lysine amino acid derivative which acts as an inhibitor of plasminogen activation or plasmin. Referring to FIGS. 25, 26, whole blood containing increasing doses of tranexamic acid was perfused through the microfluidic device and a relative shift in pressure curve to the left and a corresponding decrease in clotting time was observed as the dosage of drug samples was increased, as shown in graphs 229, 231 of FIGS. 25, 26, respectively. The results illustrated by graphs 229, 231 suggest that the addition of this drug prevents fibrin degradation due to plasminogen or plasmin present in blood and correspondingly promotes clotting inside the microfluidic device. Furthermore, the results illustrated in graphs 229, 231 demonstrate that the microfluidic device could potentially be used to monitor antifibrinolytic therapy in practical settings where hyperfibrinolysis arises.

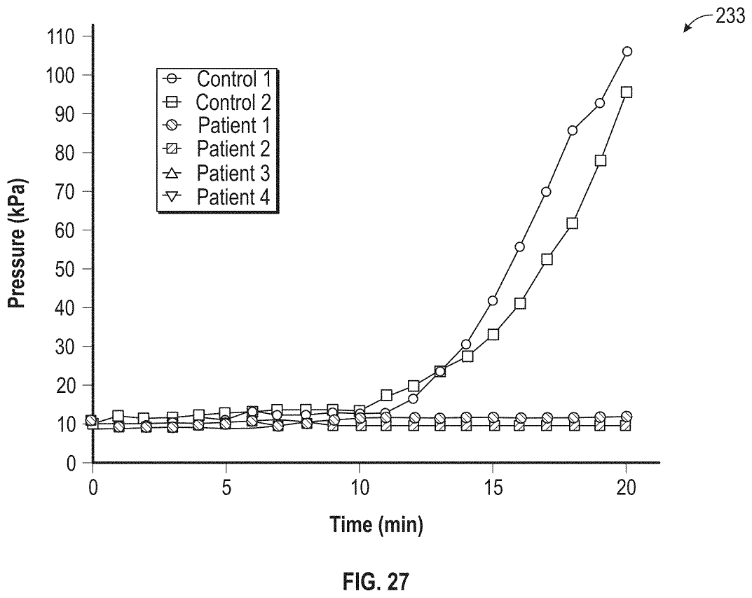

Additionally, pediatric patients on extracorporeal life support systems typically have low platelet counts, platelet dysfunction, acquired von Willebrand syndrome, hyperfibrinolysis and loss of coagulation factors. As a result, they are highly susceptible to dramatic alteration in normal hemostasis. The capability of the microfluidic device to measure defects in hemostasis present in blood samples from pediatric patients on ECMO receiving anticoagulation was explored. The patients were receiving heparin therapy, and were diagnosed with bleeding symptoms clinically, as well as low platelet counts. Referring to FIG. 27 No clotting was observed for a duration of 20 minutes when blood from four pediatric ECMO patients was perfused within the microfluidic device, clearly suggesting that, due to the combined effect of disease, surgery, and therapy, the hemostasis of the ECMO patients was significantly abnormal, as shown in graph 233 of FIG. 27. In contrast, healthy subjects exhibited increase in pressure and thrombosis within the device. The data captured in graph 233 suggests the microfluidic device may be used to test patients in critical care with altered hemostasis due to, for example, the combined effects of anticoagulation and/or other likely acquired symptoms due to operating procedures.

The experimental testing described above was designed with the hypothesis that physiological relevance of a platform that measures blood clotting may potentially serve as a more reliable point-of-care device. Several prior studies have illustrated that coagulation tests, such as the ACT and aPTT, which do not include a physiologically-relevant microenvironment (for example, fluid shear or fluid shear gradients), have limited predictive power. Moreover, although viscoelastic methods, such as TEG and ROTEM, do assess whole blood clotting, the thrombus measured are still mostly activated using artificial reagents, such as kaolin and phospholid. Conversely, the testing pertaining to the microfluidic device described above introduces a novel and underexplored hemodynamic parameter—tortuosity. The tortuosity-activated microfluidic device described herein provides several potential advantages over other tests used to monitor hemostasis and thrombosis. For example, due to the integration of complex vascular architecture (stenosis and tortuosity) in the microfluidic device, clotting inside the microfluidic device is activated by a pathophysiologically-relevant fluid mechanical environment and includes complex blood rheology that is critical to thrombosis. Additionally, when connected to a pressure sensor, blood clots can be quantified using a system comprising the microfluidic device (e.g., system 10 shown in FIGS. 1, 2). Further, the clotting time derived from the pressure curve (e.g., a pressure curve outputted by sensor 60 of system 10 shown in FIG. 1) can detect differences in blood spiked with clinically-relevant doses of anticoagulants (thrombin inhibitors) and antifibrinolytics (tranexamic acid) in-vitro, and the microfluidic device shows sensitivity to platelet count under stenosed and tortuous flow, making the microfluidic device potentially well suited to explore whether anticoagulants, antifibrinolytics and anti-platelet drug candidates that produce different behaviors in clotting dynamics. Testing of the microfluidic device described above also confirmed that pediatric patients on ECMO as well as anticoagulants did not clot within the microfluidic device. Therefore, the data presented above provides a proof-of-feasibility that the microfluidic device may be used to identify changes in hemostasis at bedside and guide therapy. Generally, the microfluidic device may be used as a tool to diagnose clotting disorders and guide therapy where thrombotic or antithrombotic drugs are administered.

The experimental microfluidic described above was fabricated from PDMS soft lithography to a size that would fit on a standard (e.g., 75 mm×50 mm) microscope slide. The cured PDMS from the master mold was bonded to a 500 mm-thick PDMS-coated glass slide and punched with inlet and outlet 1.5 mm holes. Next, the device was coated with rat tail type I collagen at 100 μg/ml infused into the microfluidic device which was pre-treated with silane. The microfluidic device was incubated at 37° C. for three to four hours and washed with saline solution prior to blood perfusion. At the inlet side of the microfluidic device, a reservoir was created by force-fitting an open slip-tip syringe and fresh blood was stored therein. At the outlet, tubing was connected to an inline, disposable pressure sensor configured to determine pressure change across the microfluidic device. The other end of the pressure sensor was connected to a syringe pump which pulled the stored blood at a mean arteriole wall shear rate of 1200 1/s. Thrombus formation was observed using time-lapse imaging (10×, NA 0.35) of fluorescently labelled fibrinogen (15 μg/ml, Alexa Fluor 647, Invitrogen) and platelets (10 μl/ml Human CD41-PE (clone: VIPL3), Invitrogen) added directly to the blood and incubated at room temperature for approximately eight minutes for whole blood imaging. Fluorescence microscopy of fibrin and platelets was then performed at an interval of approximately 30 seconds.

While exemplary embodiments have been shown and described, modifications thereof can be made by one skilled in the art without departing from the scope or teachings herein. The embodiments described herein are exemplary only and are not limiting. Many variations and modifications of the systems, apparatus, and processes described herein are possible and are within the scope of the disclosure. For example, the relative dimensions of various parts, the materials from which the various parts are made, and other parameters can be varied. Accordingly, the scope of protection is not limited to the embodiments described herein, but is only limited by the claims that follow, the scope of which shall include all equivalents of the subject matter of the claims. Unless expressly stated otherwise, the steps in a method claim may be performed in any order. The recitation of identifiers such as (a), (b), (c) or (1), (2), (3) before steps in a method claim are not intended to and do not specify a particular order to the steps, but rather are used to simplify subsequent reference to such steps.

What is claimed is:

1. A system for evaluating an ability of a blood sample to coagulate, clot, and/or occlude, the system comprising:
a microfluidic device comprising a body and a tortuous microchannel formed in the body, wherein the tortuous microchannel comprises a plurality of alternating curves and has a tortuosity index (TI) of at least 2.0; and
a pump fluidically configured to fluidically connect to the tortuous microchannel of the microfluidic device and to induce a blood flow through the tortuous microchannel at at least one of a constant pressure and a constant flowrate.

2. The system of claim 1, further comprising:
a sensor configured to fluidically connect to the tortuous microchannel of the microfluidic device and configured to determine at least one of the pressure and the flowrate induced by the pump; and
a controller configured to determine a clotting time of the blood flow based on at least one of the pressure and the flowrate of the blood flow determined by the sensor.

3. The system of claim 2, wherein the clotting time determined by the controller corresponds to a time period at which the pressure of the blood flow is between 2.3 times and 3.0 times greater than an initial pressure of the blood flow at an initiation of the blood flow induced by the pump.

4. The system of claim 1, wherein the tortuous microchannel of the microfluidic device is sinusoidal and has a frequency between 2.0 and 2.5 cycles per millimeter (mm).

5. The system of claim 4, wherein the tortuous microchannel of the microfluidic device has an amplitude of between 2 mm and 5 mm.

6. The system of claim 1, wherein the tortuous microchannel of the microfluidic device has an arc angle between the alternating curves thereof between 30° and 40°.

7. The system of claim 1, wherein the tortuous microchannel has a cross-sectional flow area between 12,000 square micrometers (μm²) and 20,000 μm².

8. The system of claim 1, wherein the microfluidic device further comprises an inlet reservoir and an inlet microchannel extending from the inlet reservoir each formed in the body, and an outlet microchannel extending to an outlet reservoir each formed in the body thereof, wherein the tortuous microchannel is positioned between the inlet microchannel and the outlet microchannel.

9. The system of claim 8, wherein the microfluidic device comprises a plurality of inlet microchannels, tortuous microchannels, and outlet microchannels extending in parallel between the inlet reservoir and the outlet reservoir.

10. The system of claim 1, wherein the tortuous microchannel is coated with at least one of a thrombus forming agent, a reagent, collagen, and a thrombus inhibiting agent, a platelet activating material, a platelet inhibiting material, a fibrin network forming material, a fibrin network disrupting material, and/or cells.

11. The system of claim 1, wherein the pump is configured to induce the blood flow at a flowrate of between 50 microliters per minute (μl/min) and 100 μl/min.

12. A system for evaluating an ability of a blood sample to coagulate, clot, and/or occlude, the system comprising:
a microfluidic device comprising a body and a tortuous microchannel formed in the body, wherein the tortuous microchannel comprises a plurality of alternating curves having an arc angle between the alternating curves between 30° and 40°; and
a pump fluidically configured to fluidically connect to the tortuous microchannel of the microfluidic device and to induce a blood flow through the tortuous microchannel at at least one of a constant pressure and a constant flowrate.

13. The system of claim 12, further comprising:
a sensor configured to fluidically connect to the tortuous microchannel of the microfluidic device and configured to determine at least one of the pressure and the flowrate induced by the pump; and
a controller configured to determine a clotting time of the blood flow based on at least one of the pressure and the flowrate of the blood flow determined by the sensor.

14. The system of claim 13, wherein the clotting time determined by the controller corresponds to a time period at which the pressure of the blood flow is between 2.3 times and 3.0 times greater than an initial pressure of the blood flow at an initiation of the blood flow induced by the pump.

15. The system of claim 12, wherein the tortuous microchannel of the microfluidic device is sinusoidal and has a frequency between 2.0 and 2.5 cycles per millimeter (mm).

16. The system of claim 12, wherein the tortuous microchannel of the microfluidic device has a tortuosity index (TI) of at least 2.0.

17. A method or evaluating an ability of a blood sample to coagulate, clot, and/or occlude, the method comprising:
(a) inducing a blood flow by a pump through a tortuous microchannel formed in a body of a microfluidic device, wherein the tortuous microchannel comprises a plurality of alternating curves and has a tortuosity index (TI) of at least 2.0;
(b) monitoring at least one of a flowrate and a pressure by a sensor fluidically connected to the tortuous microchannel;
(c) determining by a controller a clotting time of the blood flow based on a change in the pressure or the flowrate of the blood flow monitored by the sensor.

18. The method of claim 17, wherein the clotting time corresponds to a time period at which the pressure of the blood flow is between 2.3 times and 3.0 times greater than an initial pressure of the blood flow at an initiation of the blood flow induced by the pump.

19. The method of claim 17, wherein (b) comprises inducing the blood flow by the pump through the tortuous microchannel at a flowrate of between 50 microliters per minute (μl/min) and 100 μl/min such that a shear rate gradient of greater than 5000 per second per millimeter (1/s/mm).

20. The method of claim 17, wherein the tortuous microchannel comprises a plurality of alternating curves having an arc angle between the alternating curves between 30° and 40°.
